(19) United States
(12) Patent Application Publication
(10) Pub. No.: US 2018/0353335 A1
(43) Pub. Date: Dec. 13, 2018

(54) WOUND TREATMENT DEVICE FOR TREATING A SKIN WOUND AND A METHOD OF TREATING A SKIN WOUND

(71) Applicant: Dale Norman WALKER, Bilgola (AU)

(72) Inventor: Dale Norman WALKER, Bilgola (AU)

(21) Appl. No.: 15/774,063

(22) PCT Filed: Nov. 9, 2016
(86) PCT No.: PCT/AU2016/000374

§ 371 Date: May 7, 2018

(30) Foreign Application Priority Data
Nov. 9, 2015 (AU) 2015904584

(51) Int. Cl. A61F 13/02; A61F 13/00

(52) U.S. Cl. CPC A61F 13/023; A61F 13/00063; A61F 13/0213; A61F 13/0206

(57) ABSTRACT

A wound treatment device for treating a skin wound comprises a strip (100) having an upper face and a lower face. The strip (100) comprises a central portion (110) adapted to substantially extend over the wound and a pair of opposing end portions (112a and 112b) connected to the central portion (110) by way of a pair of respective opposing intermediate portions (114a and 114b) such that the central portion (110) overlaps the pair of end portions (112a and 112b). The device further comprises an adhesive (116) disposed on the lower face of the strip (100) at the end portions (112a and 112b), allowing the end portions (112a and 112b) to be adhered to skin around the wound. The strip (100) is deformable, under application of force to the upper face at the central portion (110) in a direction normal to the upper face and towards the lower face, from a self-supporting pre-use configuration in which there is a gap (118) between the pair of end portions (112a and 112b), to an in-use configuration in which the gap (118) between the pair of end portions (112a and 112b) is reduced. The strip (100) further comprises means for securing the central portion in relation to the end portions (112a and 112b) in the in-use configuration.

### FIELD

The present invention relates to wound treatment, and in particular relates to a wound treatment device for treating a skin wound and a method of treating a skin wound. The invention more particularly relates to, but is not limited to, a treatment and device for closing an open skin wound.

### BACKGROUND

Wound closure strips are known. They are a form of protective dressing designed to assist healing of a skin wound. Known wound closure strips, for example a steri-strip, is typically a flat, rectangular strip of adhesive bandage which is adhered across the wound. The strip holds the wound in place whilst providing protection for the wound from the external environment.

Known wound closure strips typically require the user to use both hands to apply the strip. One hand is required to firstly draw the skin on opposing sides of the wound closer together and hold the skin in place. The other hand is then required to tension and adhere the strip across the wound. This is often a time-consuming and cumbersome process, particularly when multiple strips are needed, for example to treat a large gash or laceration. The application of known adhesive bandages also typically requires the use of both hands to avoid the risk of the bandage from adhering to itself and being rendered unusable.

### OBJECT

It is the object of the present invention to substantially overcome or at least ameliorate one or more of the above disadvantages.

### SUMMARY OF THE INVENTION

Accordingly, one aspect of the present invention provides a wound treatment device for treating a skin wound, the device comprising:

a strip having an upper face and a lower face, the strip comprising:
a central portion adapted to substantially extend over the wound; and
a pair of opposing end portions connected to the central portion by way of a pair of respective opposing intermediate portions such that the central portion overlaps the pair of end portions; and

an adhesive disposed on the lower face of the strip at the end portions, allowing the end portions to be adhered to skin around the wound;

wherein the strip is deformable, under application of force to the upper face at the central portion in a direction normal to the upper face and towards the lower face, from a self-supporting pre-use configuration in which there is a gap between the pair of end portions, to an in-use configuration in which the gap between the pair of end portions is reduced;

further wherein the strip further comprises means for securing the central portion in relation to the end portions in the in-use configuration.

In a preferred form, the strip is integrally formed.

In a preferred form, each of the intermediate portions forms an acute angle bend with the central portion.

In a preferred form, each of the intermediate portions forms an acute angle bend with the respective end portion.

The means for securing the central portion in relation to the end portions may comprise an adhesive disposed on the lower face of the strip at the central portion.

Alternatively or additionally, the means for securing the central portion in relation to the end portions comprises an adhesive disposed on the lower face of the strip at the intermediate portions.

Alternatively or additionally, the means for securing the central portion in relation to the end portions comprises an adhesive disposed on the upper face of the strip at the intermediate portions.

In a preferred form, the adhesive extends at least substantially across the entire lower face of the strip, thereby forming the means for securing the central portion.

In a preferred form, the wound treatment device further comprises a backing mounted on the adhesive.

In a preferred form, the backing includes a gripping tab.

In a preferred form, the gripping tab extends generally transverse to an adjacent region of the lower face of the strip.

The means for securing the end portions in relation to the central portion may alternatively comprise a locking element disposed on each of the end portions, each locking element being adapted to engage a corresponding aperture in the respective intermediate portion when the strip is in the in-use configuration.

The strip may include a pair of opposing tabs extending from opposing ends of the central portion, the tabs each including an adhesive disposed on a lower face thereof and being adapted to adhere to the skin around the wound when the strip is in the in-use configuration.

Another aspect of the present invention provides a method of treating a skin wound, the method comprising:

positioning a central portion of a strip in a pre-use configuration over the wound, such that the central portion substantially extends over the wound;

adhering a pair of opposing end portions of the strip to skin on opposing sides of the wound, such that there is a gap between the end portions;

applying force to the central portion in a direction towards the wound, the force deforming the strip into an in-use configuration, in which the gap between the end portions is reduced, thereby drawing the skin on the opposing sides of the wound towards each other; and

securing the central portion in relation to the end portions in the in-use configuration.

### BRIEF DESCRIPTION OF THE DRAWINGS

Preferred embodiments of the invention will now be described, by way of example only, with reference to the accompanying drawings in which:

FIG. 1 is a front perspective view of a first embodiment of a wound treatment device, in a pre-use configuration;

FIG. 2 is a front view of the wound treatment device shown in FIG. 1;

FIG. 3 is a front perspective view of the wound treatment device shown in FIG. 1, in an in-use configuration;

FIG. 4 is a front view of the wound treatment device shown in FIG. 1, in the in-use configuration;

FIG. 5 is a front perspective view of the wound treatment device shown in FIG. 1, in the pre-use configuration and during an initial stage of application;

FIG. 6 is a front perspective view of the wound treatment device shown in FIG. 1, in the pre-use configuration and during an intermediate stage of application;

FIG. 7 is a front perspective view of the wound treatment device shown in FIG. 1, in the pre-use configuration and during a final stage of application;

FIG. 8 is a front perspective view of the wound treatment device shown in FIG. 1, in the in-use configuration;

FIG. 9 is a perspective view of a second embodiment of a wound treatment device;

FIG. 10 is a perspective view of a third embodiment of a wound treatment device;

FIG. 11 is a perspective view of a fourth embodiment of a wound treatment device;

FIG. 12 is a front view of the wound treatment device shown in FIG. 1, showing the adhesive disposed only on the lower face at the end portions and the lower face at the central portion;

FIG. 13 is a front view of the wound treatment device shown in FIG. 1, showing the adhesive disposed only on the lower face at the end portions and the lower face at the intermediate portions; and

FIG. 14 is a front view of the wound treatment device shown in FIG. 1, showing the adhesive disposed only on the lower face at the end portions and the upper face at the intermediate portions.

FIG. 15 is a perspective view of the wound treatment device shown in FIG. 1, showing an additional adhesive covering.

FIG. 16 is a front perspective view of the wound treatment device shown in FIG. 1, showing a plurality of creases formed on the central portion; and

FIG. 17 is a front view of the wound treatment device shown in FIG. 1, showing the backing having a gripping tab.

### DESCRIPTION OF EMBODIMENTS

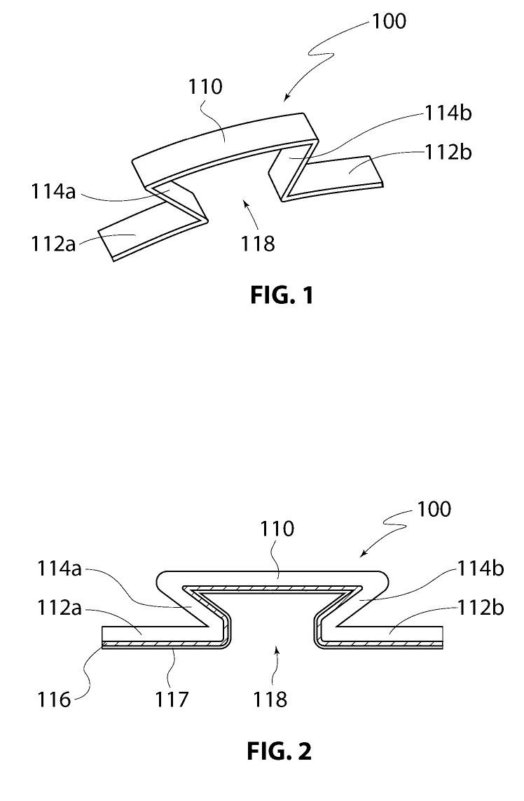

FIGS. 1 and 2 show a first embodiment of a wound treatment device for treating a skin wound. As will be discussed in further detail below, the wound treatment device is primarily for closing an open skin wound, but is not limited to this particular application and may also be used to treat a closed skin wound. The wound treatment device includes a strip 100 that forms the body of the device. The strip 100 is in the form of a single strip of material. Suitable materials include woven fabric, plastic (for example PVC, polyethylene or polyurethane), latex, elastic and microporous materials. The strip 100 has an upper face and a lower face, and includes a central portion 110 which is adapted to extend over the wound. The strip 100 further includes a pair of opposing end portions 112a and 112b. Each of the end portions 112a and 112b is connected to the central portion 110 by a pair of respective opposing intermediate portions 114a and 114b.

As best shown in FIG. 2, the device further includes an adhesive 116 disposed on the lower face of the strip 100 at the end portions 112a and 112b. The adhesive 116 allows the end portions 112a and 112b to be adhered to skin around the wound. In the embodiment as shown in FIG. 2, the adhesive 116 extends across the entire lower face of the strip 100. The adhesive 116 may be formed of a pressure-sensitive hypoallergenic adhesive, such as acrylate (for example, methacrylates or epoxy diacrylates). The device further includes a backing 117 mounted on the adhesive 116 across the lower face of the strip 100. The backing 117 may be formed of coated paper or plastic. The backing 117 may comprise two separate parts. The strip 100 is in a pre-use configuration, in which there is a gap 118 between the pair of end portions 112a and 112b. The intermediate portions 114a and 114b each form an acute angle bend with the central portion 110. Similarly, the intermediate portions 114a and 114b each form an acute angle bend with the respective end portions 112a and 112b. In this configuration, the strip 100 is self-supporting and has sufficient stiffness such that it does not collapse on itself when it is in a free-standing form, prior to application.

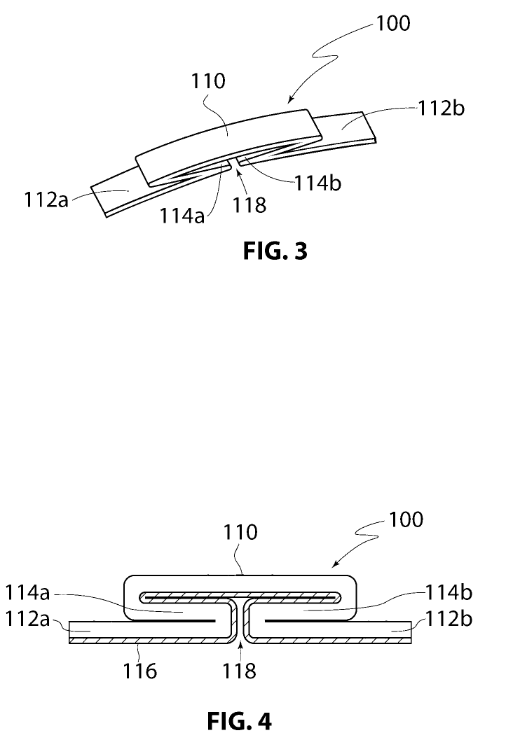

FIGS. 3 and 4 show the strip 100 in an in-use configuration. In this configuration, the gap 116 between the end portions 112a and 112b is reduced. The acute angles of the bends between the intermediate portions 114a and 114b and the central portion 110 are also reduced. Likewise, the acute angles of the bends between the intermediate portions 114a and 114b and the respective end portions 112a and 112b are reduced. The central portion 110 is also secured in relation the end portions 112a and 112b, where the central portion 110, the intermediate portions 114a and 114b, and the end portions 112a and 112b all lie flat against one other. The adhesive 116 extending across the entire lower face of the strip 100 allows the end portions 112a and 112b to be adhered to the skin around the wound, and additionally forms a means for securing the central portion 110 in relation to the end portions 112a and 112b, as will be described below. Each of the intermediate portions 114a and 114b has a length that is slightly less than half the length of the central portion 110, such that the end portions 112a and 112b do not overlap with each other when the strip 100 is in the in-use configuration.

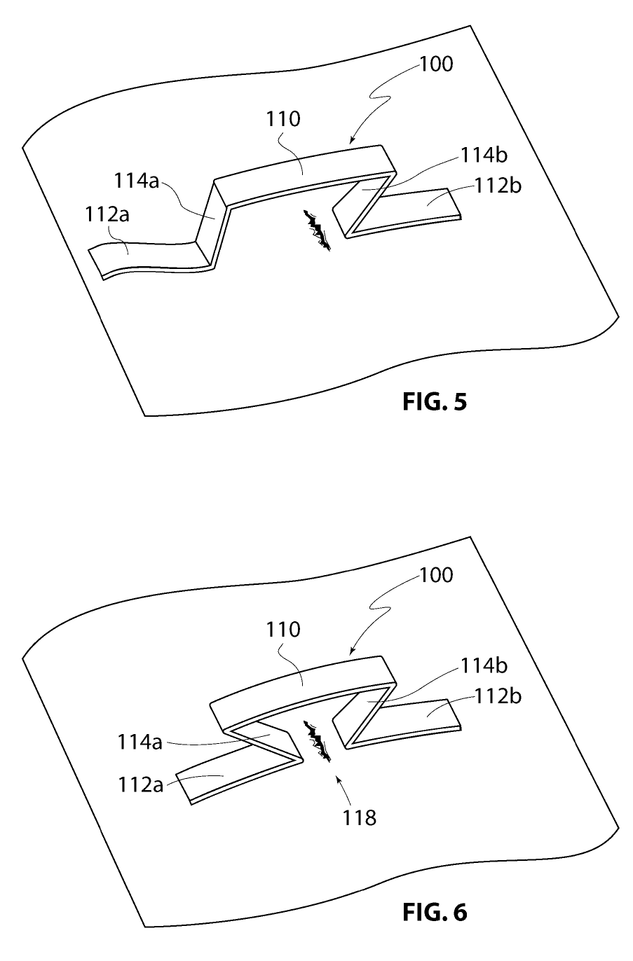

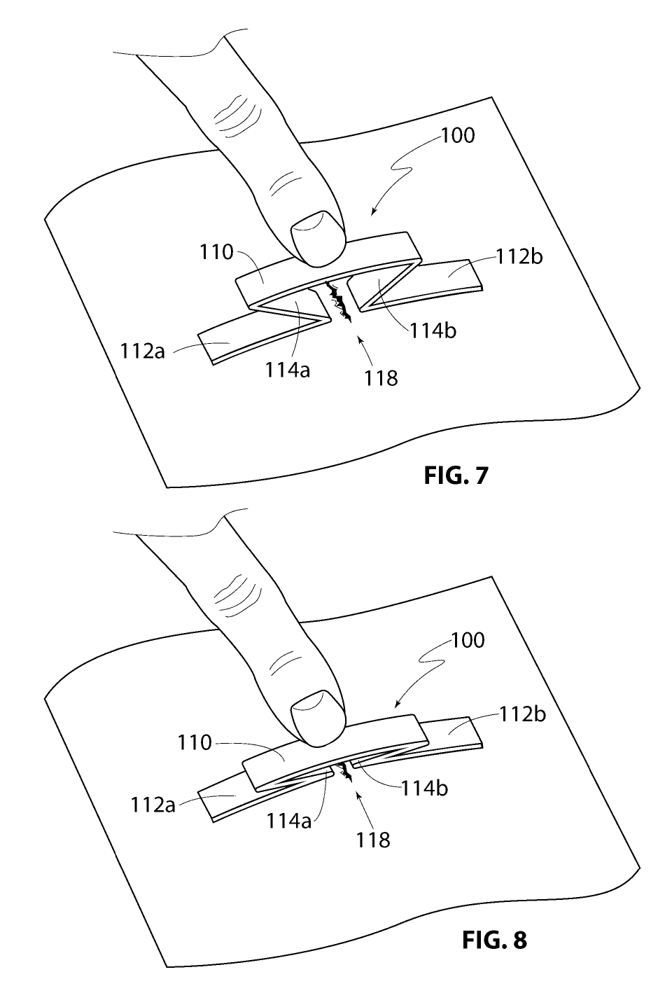

The application of the wound treatment device will now be described with reference to FIGS. 5 to 8. The end portion 112b is adhered to skin on one side of the wound, and the other end portion 112a is adhered to an opposing side of the wound, thereby creating the gap 118 between the end portions 112a and 112b. In the process, the central portion 110 of the strip 100 is positioned over the wound such that the central portion 110 substantially extends over the wound. The adhesion of the end portions 112a and 112b to the skin on opposing sides of the wound may be done sequentially or simultaneously.

Force is then applied to the central portion 110 in a direction towards the wound, causing the strip 100 to deform at each of the bends between the end portions 112a and 112b, the intermediate portions 114a and 114b, and the central portion 110. The intermediate portions 114a and 114b pivot about their respective bends, and the resulting force draws the end portions 112a and 112b towards each other, thereby reducing the gap 118 and flattening the strip 100. It is envisaged that when the wound treatment device is configured to close an open wound, each of the intermediate portions 114a and 114b will typically have a length that is slightly less than half the length of the central portion 110. This allows the end portions 112a and 112b to be drawn towards each other until the end portions 112a and 112b are brought into contact, thereby closing the gap 118, simultaneously drawing the skin on the opposing sides of the wound towards each other. Alternatively, when the wound treatment device is configured to treat or protect a closed wound, the length of each of the intermediate portions 114a and 114b will typically be reduced such that the skin on the opposing sides of the wound is still drawn towards each other, but to a lesser extent such that the gap 118 does not completely close. The central portion 110 is then secured in relation to the end portions 112a and 112b by way of the adhesive 116 such that the strip 100 is retained in the in-use configuration. In the embodiment as shown in FIG. 4, the adhesive 116 on the lower face at the central portion 110 sticks to the adhesive 116 on the lower face of the intermediate portions 114a and 114b so as to secure the central portion 110. Advantageously, the construction of the device allows each of the end portions 112a and 112b to be adhered individually (i.e. one at a time), such that the strip 100 may be applied, deformed and secured using only one hand. This provides for a quick and relatively simple way to treat a skin wound.

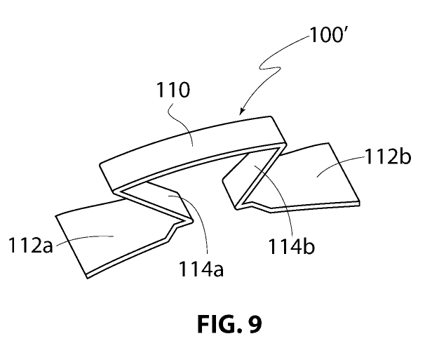

FIG. 9 shows a second embodiment of a wound treatment device which is generally identical to the first embodiment of the wound treatment device described above, except that each of the end portions 112a and 112b of the strip 100′ has a larger face in this embodiment. This in turn increases the surface area of the adhesive 116 which is disposed on the lower face at the end portions 112a and 112b, thus allowing for better adhesion to the skin around the wound.

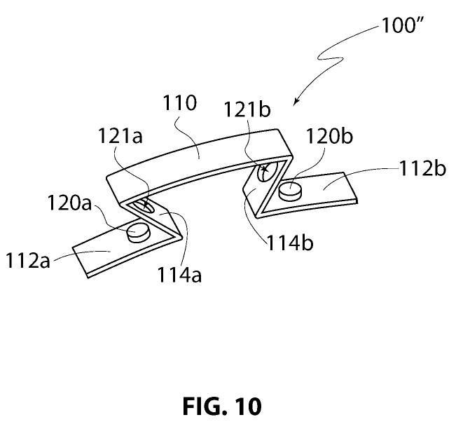

FIG. 10 shows a third embodiment of a wound treatment device which is generally identical to the first embodiment of the wound treatment device described above. However, the end portion 112a of the strip 100″ in this embodiment includes a first locking element 120a and the intermediate portion 114a includes a first aperture 121a corresponding to the shape of the first locking element 120a. Likewise, the end portion 114b of the strip 100″ includes a second locking element 120b and the intermediate portion 114b includes an aperture 121b corresponding to the shape of the second locking element 120b. The apertures 121a and 121b are each adapted to receive the respective locking element 120a and 120b therein in a snap-fitting engagement. When the strip 100″ is deformed from the pre-use configuration to the in-use configuration, the locking elements 120a and 120b each engage their respective apertures 121a and 121b and adhere to the lower face of the strip 100″ at the central portion 110. The locking plugs 120a and 120b therefore allow the end portions 112a and 112b to be secured to the central portion 110 in the in-use configuration. This allows shape of the strip 100 to be better maintained whilst in the in-use configuration.

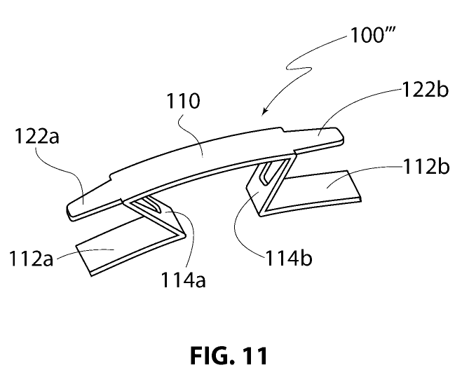

FIG. 11 shows a fourth embodiment of a wound treatment device, which is again generally identical to the first embodiment of the wound treatment device described above. However, the strip 100′ in this embodiment includes a pair of opposing tabs 122a and 122b extending from the opposing ends of the central portion 110. The tabs 122a and 122b are cut out from the intermediate portions 114a and 114b, respectively. The tabs 122a and 122b also include an adhesive disposed at their lower faces. When the strip 100′ is deformed from the pre-use configuration to the in-use configuration, the tabs 122a and 122b are each brought into contact with and adhered to the underlying skin on the opposing sides, thereby enhancing the adhesion of the strip 100″′ to the skin on the opposing sides of the wound and allowing the shape of the strip 100″′ to be better maintained whilst in the in-use configuration.

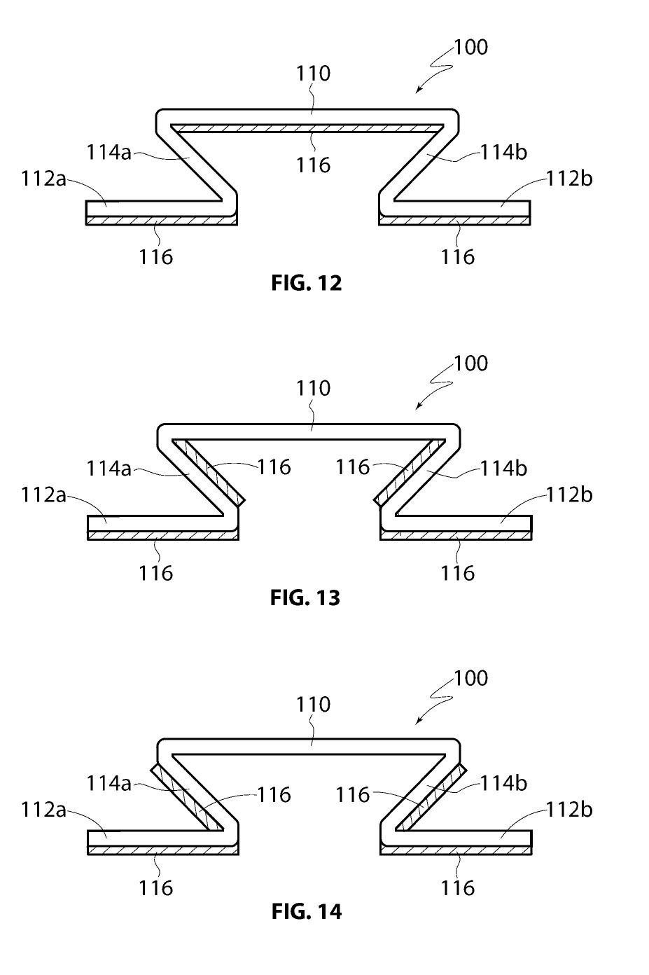

A person skilled in the art will appreciate various other possible modifications and additions to the wound treatment device described. It is envisaged that alternate means may be used to secure the central portion 110 in relation to the end portions 112a and 112b. When the means is the adhesive 116, it is envisaged that the adhesive 116 may disposed on the lower face at the central portion 110 so as to adhere the central portion 110 is to the intermediate portions 114a and 114b, as shown in FIG. 12. Alternatively or additionally, the adhesive 116 may be disposed on the lower face at the intermediate portions 114a and 114b so as to adhere the intermediate portions 114a and 114b to the central portion 110, as shown in FIG. 13. Alternatively or additionally, the adhesive may be disposed on the upper face at the intermediate portions 114a and 114b so as to adhere the intermediate portions 114a and 114b to the end portions 112a and 112b, as shown in FIG. 14.

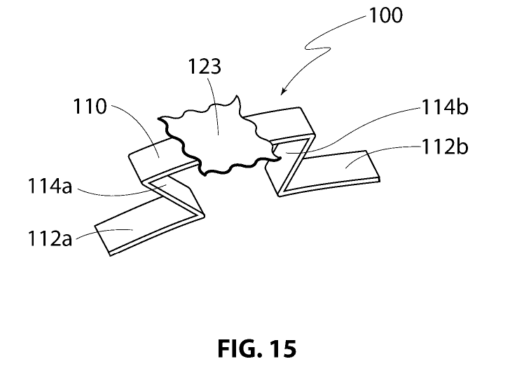

It is also envisaged that the wound treatment device may include an adhesive covering 123 (as shown in FIG. 15) which is adhered to, or integrally formed with, the upper face of the strip 100 at the central portion 110. The adhesive covering 123 is wider than the central portion 110, effectively extending the upper face of the strip 100 at the central portion 110. When the strip 100 is deformed from the pre-use configuration to the in-use configuration, the adhesive covering 123 is brought into contact with and adhered to the underlying skin across the width of the strip 100. This enhances the adhesion of the strip 100 to the skin across the wound and also allows the shape of the strip 100 to be better maintained whilst in the in-use configuration.

The device may also include a metal strip embedded in the strip 100 to increase stiffness and assist in maintaining the shape of the strip 100 whilst being deformed and in the in-use configuration. The metal strip would be plastically deformed at the bends. The strip 100 may also have a corrugated or a ribbed transverse cross-section to improve stiffness. The strip 100 may also include polyester filaments to improve strength and stiffness. The strip 100 may have width and length dimensions ranging from 3 to 24 mm (width) and 37 to 125 mm (length). It is envisaged that the thickness of the strip 100 may also be varied to improve stiffness. For example, the central portion 110 may be thicker than the intermediate portions 114a and 114b and the end portions 112a and 112b. This better allows the shape of the central portion 110 to be maintained whilst the strip 100 is being deformed. The increased thickness of the central portion 110 may also provide added protection for the wound against further damage from physical contact. The strip 100 may be waterproof or airtight, so as to provide an occlusive dressing. The strip 100 may also be formed of a translucent or transparent material to assist with the positioning and alignment of the device over the wound by enabling a user to view the location of the wound through the strip 100 during application.

The device may alternatively include an absorbent pad disposed on the lower face of the strip 100 at the central portion 110, particularly in the embodiments where the adhesive is disposed only on the lower and/or upper faces at the intermediate portions 114a and 114b, as shown in FIGS. 13 and 14. The absorbent pad may be sized so that it covers only a small middle section of the central portion 110 directly overlying the gap 118. In these embodiments, the strip 100 may be dimensioned such that the gap 118 between the end portions 112a and 112b is larger, so as to allow the absorbent pad to contact the wound when the strip 100 is in the in-use configuration. It is envisaged that the gap 118 may have length and/or width dimensions of 20 mm or more, with the absorbent pad having similar dimensions to allow for complete coverage over a minor skin wound. In this arrangement, the lengths of the intermediate portions 114a and 114b are reduced (to around 10 mm, for example) such that when the strip 100 is deformed from the pre-use configuration to the in-use configuration, the extent to which the end portions 112a and 112b (and the underlying skin) is drawn together also decreases. This results in only a small reduction in the size of the gap 118, allowing the strip 100 to be applied to skin wounds which do not require closing, in place of a regular adhesive bandage. The absorbent pad may be formed of cotton, preferably with a thin, porous-polymer coating so as to prevent the pad from sticking to the wound. The absorbent pad may be medicated with an antiseptic solution. The absorbent pad may alternatively be formed of a water-absorbing gel.

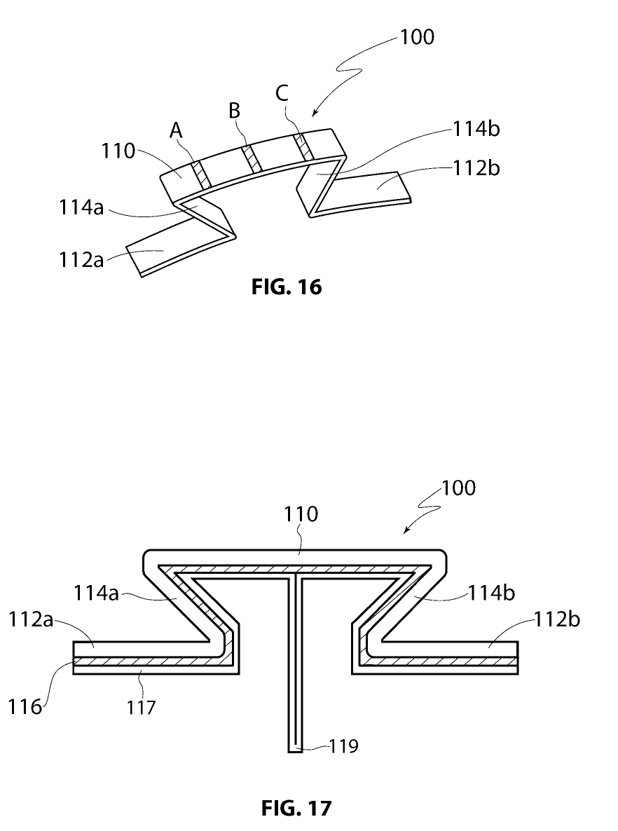

It is further envisaged that when the strip 100 is relatively stiff, one or more creases A, B and/or C may be formed on the central portion 110, as shown in FIG. 16. The creases A, B and/or C would increase the pliability of the strip 100, so that the device may more readily conform to the shape profile of the surface on which the device is being applied. The creases A, B and/or C each effectively form a hinge. It is envisaged that other forms of hinges may also be utilised as an alternative to the creases A, B and/or C.

The backing 117 may have a gripping tab 119 which extends away from the lower face at the central portion 110, as shown in FIG. 17. The backing 117 may be folded over itself at the middle, or provided in a two-part form, so as to create the gripping tab 119. The gripping tab 119 facilitates quick and easy removal of the backing 117 by allowing the user to simply pinch the gripping tab 119 and pull the entire backing 117 away from the adhesive 116 in one motion. The backing 117 and gripping tab 119 may be applied to any one of the embodiments of the device as described above. It is envisaged that when the backing 117 is applied to the fourth embodiment as shown in FIG. 11, the backing 117 may also extend across the lower face of the tabs 122a and 122b.

1. A wound treatment device for treating a skin wound, the device comprising:
a strip having an upper face and a lower face, the strip comprising:
a central portion adapted to substantially extend over the wound; and a pair of opposing end portions connected to the central portion by way of a pair of respective opposing intermediate portions such that the central portion overlaps the pair of end portions; and
an adhesive disposed on the lower face of the strip at the end portions, allowing the end portions to be adhered to skin around the wound;
wherein the strip is deformable, under application of force to the upper face at the central portion in a direction normal to the upper face and towards the lower face, from a self-supporting pre-use configuration in which there is a gap between the pair of end portions, to an in-use configuration in which the gap between the pair of end portions is reduced;
further wherein the strip further comprises means for securing the central portion in relation to the end portions in the in-use configuration.

2. The wound treatment device of claim 1, wherein the strip is integrally formed.

3. The wound treatment device of claim 1, wherein each of the intermediate portions forms an acute angle bend with the central portion.

4. The wound treatment device of claim 1, wherein each of the intermediate portions forms an acute angle bend with the respective end portion.

5. The wound treatment device of claim 1, wherein the means for securing the central portion in relation to the end portions comprises an adhesive disposed on the lower face of the strip at the central portion.

6. The wound treatment device of claim 1, wherein the means for securing the central portion in relation to the end portions comprises an adhesive disposed on the lower face of the strip at the intermediate portions.

7. The wound treatment device of claim 1, wherein the means for securing the central portion in relation to the end portions comprises an adhesive disposed on the upper face of the strip at the intermediate portions.

8. The wound treatment device of claim 1, wherein the adhesive extends at least substantially across the entire lower face of the strip, thereby forming the means for securing the central portion.

9. The wound treatment device of claim 1, further comprising a backing mounted on the adhesive.

10. The wound treatment device of claim 9, wherein the backing includes a gripping tab.

11. The wound treatment device of claim 10, wherein the gripping tab extends generally transverse to an adjacent region of the lower face of the strip.

12. The wound treatment device of claim 1, wherein the means for securing the end portions in relation to the central portion comprises a locking element disposed on each of the end portions, each locking element being adapted to engage a corresponding aperture in the respective intermediate portion when the strip is in the in-use configuration.

13. The wound treatment device of claim 1, wherein the strip includes a pair of opposing tabs extending from opposing ends of the central portion, the tabs each including an adhesive disposed on a lower face thereof and being adapted to adhere to the skin around the wound when the strip is in the in-use configuration.

14. A method of treating a skin wound, the method comprising:
positioning a central portion of a strip in a pre-use configuration over the wound, such that the central portion substantially extends over the wound;
adhering a pair of opposing end portions of the strip to skin on opposing sides of the wound, such that there is a gap between the end portions;
applying force to the central portion in a direction towards the wound, the force deforming the strip into an in-use configuration, in which the gap between the end portions is reduced, thereby drawing the skin on the opposing sides of the wound towards each other; and
securing the central portion in relation to the end portions in the in-use configuration.
